(19) United States
(12) Patent Application Publication
(10) Pub. No.: US 2018/0055840 A1
(43) Pub. Date: Mar. 1, 2018

(54) ORAL FORMULATION

(71) Applicant: OTSUKA PHARMACEUTICAL CO., LTD., Tokyo (JP)

(72) Inventors: Taro IWAMOTO, Osaka (JP); Nobuyuki KURAHASHI, Osaka (JP); Yoshikazu OKA, Osaka (JP); Chikako TAKEDA, Osaka (JP)

(73) Assignee: OTSUKA PHARMACEUTICAL CO., LTD., Tokyo (JP)

(21) Appl. No.: 15/795,500

(22) Filed: Oct. 27, 2017

Related U.S. Application Data
(63) Continuation of application No. 15/479,409, filed on Apr. 5, 2017.
Application No. 15/479,409 is a continuation of application No. 14/397,683, filed on Oct. 29, 2014, now abandoned.
(60) Provisional application No. 61/640,474, filed on Apr. 30, 2012.
(60) Provisional application No. 61/783,163, filed on Mar. 14, 2013.

(51) Int. Cl. A61K 31/496; A61K 47/42; A61K 47/36; A61K 9/00; A61K 47/26

(52) U.S. Cl. CPC A61K 31/496; A61K 47/42; A61K 47/26; A61K 9/0053; A61K 9/0056; A61K 47/36

(57) ABSTRACT

Provided are an oral formulation capable of improving easy administrability and showing good preservation stability, and a substrate for oral formulation. An oral formulation containing a medicament; sugar alcohol; one or more kinds of hydrophilic polysaccharides selected from the group consisting of acacia, pullulan and maltodextrin; a gelling agent; and water, and a substrate for oral formulation, which contains sugar alcohol; the above-mentioned hydrophilic polysaccharides; a gelling agent; and water.

### TECHNICAL FIELD OF THE INVENTION

The present invention relates to an oral formulation capable of improving easy administrability, and a substrate for oral formulation.

### BACKGROUND OF THE INVENTION

Easiness of administration of an oral formulation is one of the important factors of pharmacotherapy. For example, oral formulations such as powder, tablet and the like are sometimes difficult to take because of the dosage, size and the like of the formulation. In some cases, the taste of a medicament, particularly an uncomfortable taste such as a bitter taste and the like, smell and the like cause refusal of medicament intake. Since the administrability of the formulation can prevent treatment of diseases, an oral formulation which is easy to take is desired.

### DOCUMENT LIST

### Patent Document

patent document 1: JP-A-2006-316052

### SUMMARY OF THE INVENTION

### Problems to be Solved by the Invention

The present inventors have found that an oral formulation containing a medicament, sugar alcohol, a gelling agent, and water can be easily taken and can improve medication adherence. However, they have found that the formulation is associated with a problem of precipitation of sugar alcohol depending on the kind and/or content ratio thereof, in the formulation or on the surface thereof, during preservation of the formulation.

It is therefore an object of the present invention to provide an oral formulation that can be taken easily, can improve medication adherence, and shows good preservation stability.

### Means of Solving the Problems

The present inventors have conducted intensive studies in an attempt to achieve the aforementioned object and found that the precipitation of sugar alcohol during preservation of the formulation can be suppressed by adding one or more kinds of hydrophilic polysaccharides selected from the group consisting of acacia, pullulan and maltodextrin to the above-mentioned formulation, which resulted in the completion of the present invention.

Accordingly, the present invention provides the following.

[1] An oral formulation comprising a medicament; sugar alcohol; one or more kinds of hydrophilic polysaccharides selected from the group consisting of acacia, pullulan and maltodextrin; a gelling agent; and water.
[2] The oral formulation of the above-mentioned [1], wherein the sugar alcohol comprises one or more kinds selected from the group consisting of maltitol, sorbitol and xylitol.
[3] The oral formulation of the above-mentioned [2], wherein the sugar alcohol comprises maltitol, sorbitol and xylitol.
[4] The oral formulation of any of the above-mentioned [1]-[3], wherein one or more kinds of hydrophilic polysaccharides selected from the group consisting of acacia, pullulan and maltodextrin comprise at least maltodextrin.
[5] The oral formulation of any of the above-mentioned [1]-[4], wherein the content of one or more kinds of hydrophilic polysaccharides selected from the group consisting of acacia, pullulan and maltodextrin is 0.1-10 wt %.
[6] The oral formulation of any of the above-mentioned [1]-[5], wherein the gelling agent comprises at least gelatin.
[7] The oral formulation of any of the above-mentioned [1]-[6], wherein the content of water is 2-30 wt %.
[8] The oral formulation of any of the above-mentioned [1]-[7], wherein the content of sugar alcohol is 50-95 wt %.
[9] The oral formulation of any of the above-mentioned [1]-[8], wherein the content of the gelling agent is 1-20 wt %.
[10] The oral formulation of any of the above-mentioned [1]-[9], wherein the gelling agent consists only of gelatin.
[11] The oral formulation of any of the above-mentioned [1]-[10], wherein the medicament is a basic medicament.
[12] The oral formulation of the above-mentioned [11], wherein the basic medicament is 7-[4-(4-benzo[b]thiophen-4-yl-piperazin-1-yl)butoxy]-1H-quinolin-2-one or a salt thereof, or aripiprazole or a salt thereof.
[13] The oral formulation of any of the above-mentioned [1]-[12], further comprising one or more kinds of additives selected from the group consisting of a flavor, a colorant, a preservative and a pH adjuster.
[14] The oral formulation of any of the above-mentioned [1]-[13], wherein the pH is adjusted to 5-8.
[15] A substrate for oral formulation, comprising sugar alcohol; one or more kinds of hydrophilic polysaccharides selected from the group consisting of acacia, pullulan and maltodextrin; a gelling agent; and water.
[16] The oral formulation of the above-mentioned [11], wherein the basic medicament is 7-[4-(4-benzo[b]thiophen-4-yl-piperazin-1-yl)butoxy]-1H-quinolin-2-one or a salt thereof.
[17] The oral formulation of any of the above-mentioned [1]-[12], further comprising a pH adjuster.
[18] The oral formulation of the above-mentioned [17], wherein the pH adjuster is trisodium citrate dihydrate.

### Effect of the Invention

Since the oral formulation of the present invention shows good comfortableness during use, it motivates the patients to take the formulation, which in turn can improve the medication adherence. Moreover, the oral formulation of the present invention suppresses precipitation of sugar alcohol during preservation by adding one or more kinds of hydrophilic polysaccharides selected from the group consisting of acacia, pullulan and maltodextrin. According to the present invention, therefore, an oral formulation capable of affording the effects of improved medication adherence and good preservation stability can be provided.

The oral formulation of the present invention can be administered without water and is free of an uncomfortable taste and smell of the medicament when licked or crunched in the mouth, can be taken easily, and as a result of which can improve the medication adherence.

Furthermore, since the oral formulation of the present invention can be taken without water, it can be conveniently taken quickly irrespective of the place, time and the like. Moreover, since the oral formulation of the present invention can be taken without water, it is useful for patients requiring limitation of water intake due to other diseases.

A substrate for the oral formulation of the present invention is useful as a starting material of the oral formulation of the present invention.

### DESCRIPTION OF EMBODIMENTS

In the oral formulation of the present invention, the medicament is not particularly limited and, for example, anti-anxiety agents (e.g., diazepam, nitrazepam, ethyl loflazepate, clorazepate dipotassium, tofisopam, triazolam, bromazepam, oxazolam, oxazepam, cloxazolam, barbital), antiepileptic agents (e.g., phenytoin, sodium valproate, phenobarbital, nitrazepam), analgesic antipyretic agents (e.g., acetaminophen, ibuprofen, ketoprofen, indomethacin, mefenamic acid, flufenamic acid, flufenamic acid aluminum, aspirin, aspirin aluminum, ethenzamide, isopropylantipyrine, sulpyrine, diclofenac sodium, loxoprofen sodium, tiaramide hydrochloride, emorfazone, salicylamide, sasapyrine), psychoneurotic agents (e.g., perphenazine, levomepromazine, chlorpromazine hydrochloride, chlorprothixene, meprobamate, hydroxyzine hydrochloride, imipramine hydrochloride, amoxapine, sulpiride, clotiazepam, etizolam, bromvalerylurea, allylisopropylacetylurea, difenidol hydrochloride, aripiprazole), spasmolytic agents (e.g., butylscopolamine bromide, flopropione, scopolia extract, methylbenactyzium bromide, timepidium bromide, methylscopolamine bromide, scopolamine hydrobromide), cardiotonic agents (e.g., etilefrin hydrochloride, ubidecarenone, caffeine, denopamine, vesnarinone), anti-arrhythmic agents (e.g., carteolol hydrochloride, pindolol, propranolol hydrochloride, amisalin, indenolol hydrochloride, atenolol, disopyramide, mexiletine hydrochloride, verapamil hydrochloride, aprindine hydrochloride, propafenone hydrochloride, cibenzoline succinate), diuretics (e.g., spironolactone, furosemide, trichlormethiazide, polythiazide, triamterene, chlorthalidone, piretanide, metolazone, mefruside, tolvaptan, mozavaptane hydrochloride), antihypertensive agents (e.g., todralazine hydrochloride, methyldopa, rescinnamine, terazosin hydrochloride, prazosin hydrochloride, pindolol, nicardipine hydrochloride, manidipine hydrochloride, nisoldipine, nitrendipine, nilvadipine, alacepril, delapril hydrochloride, captopril, enalapril maleate), antihyperlipidemic agents (e.g., gamma oryzanol, nicomol, pravastatin sodium, simvastatin, probucol), antitussives and expectorant agents (e.g., pentoxyverine citrate, bromhexine hydrochloride, codeine phosphate, orciprenaline sulfate, salbutamol sulfate, trimetoquinol hydrochloride, ketotifen fumarate, azelastine hydrochloride, oxatomide, terfenadine, dihydrocodeine phosphate, hydrocodeine phosphate sekisanol, dextromethorphan phenolphthalinate, dextromethorphan hydrobromide, tipepidine citrate, tipepidine hibenzate, noscapine, noscapine hydrochloride, guaifenesin, potassium guaiacolsulfonate), steroids (e.g., mestanolone, prednisolone, estriol, progesterone, triamcinolone acetate, dexamethasone, betamethasone), gout remedies (e.g., allopurinol, colchicine, probenecid), antidiabetic agents (e.g., buformin hydrochloride, tolbutamide, gliclazide), antihistamic agents (e.g., clemastine fumarate, clemastine maleate, diphenhydramine hydrochloride, diphenhydramine salicylate, diphenhydramine tannate, d-chlorpheniramine maleate, chlorpheniramine maleate, mequitazin, triprolidine hydrochloride, dimethindene maleate, alimemazine tartarate, meclizine hydrochloride, dimenhydrinate, promethazine hydrochloride, carbinoxamine maleate, diphenylpyraline hydrochloride), anti-allergic agents (e.g., tranilast, tranexamic acid, ketotifen fumarate, repirinast, oxatomide, sodium cromoglicate, glycyrrhetinic acid, glycyrrhizin acid, glycyrrhizinate dipotassium, ammonium glycyrrhizinate, monoammonium glycyrrhizinate, methylephedrine hydrochloride, phenylpropanolamine hydrochloride, phenylephrine hydrochloride, naphazoline hydrochloride, tetryzoline, methoxyphenamine hydrochloride), peptic ulcer remedies (cetraxate hydrochloride, sofalcone, teprenone, irsogladine maleate, rebamipide, cimetidine, famotidine, ranitidine hydrochloride, omeprazole), smoking-cessation aids (e.g., nicotine), agents for dental and oral use (e.g., cetylpyridinium chloride, sodium azulene sulfonate, dequalinium hydrochloride, platycodon extract, camomile extract, chlorhexidine hydrochloride), cerebral infarction sequelae improving agents (e.g., dihydroergotoxine mesylate), bronchodilator agents (aminophylline, diprophylline, theophylline, proxyphylline, procaterol hydrochloride hydrate), antacids (synthetic aluminum silicate, synthetic hydrotalcite, sodium hydrogen carbonate, precipitated calcium carbonate, magnesium aluminometasilicate, magnesium oxide, magnesium carbonate, magnesium hydroxide, aluminum hydroxide gel), acid agents (betaine hydrochloride, glutamic acid hydrochloride), gastrointestinal function regulators (carnitine chloride, bethanechol chloride), constipating agents (berberine chloride, berberine tannate, bismuth subnitrate, bismuth subgallate, albumin tannate), mucosal repair agents (aldioxa, sodium copper chlorophyllin, potassium copper chlorophyllin, methylmethionine sulfonium chloride), laxative agents (sennoside, sennoside A•B, bisacodyl, phenovalin, phenolphthalein, dioctyl sodium sulfosuccinate), anthelmintic antiprotozoal agents (santonin, metronidazole), vitamins (retinol acetate, liver oil, ergocalciferol, alfacalcidol, thiamine hydrochloride, thiamine sulfate, fursultiamine, octotiamine, riboflavin, pyridoxine hydrochloride, nicotinic acid, calcium pantothenate, cobamamide, biotin, ascorbic acid, tocopherol acetate, menatetrenone), antiplatelet agents (e.g., cilostazol), therapeutic agents for carnitine deficiency (levocarnitine, levocarnitine chloride), 7-[4-(4-benzo[b]thiophen-4-yl-piperazin-1-yl)butoxy]-1H-quinolin-2-one (hereinafter to be referred to as compound (I)), and the like can be mentioned.

Examples of the basic medicament to be used for the oral formulation of the present invention include compound (I) or a salt thereof, and aripiprazole or a salt thereof. Compound (I) and a salt thereof can be produced by the method described in JP-A-2006-316052, or a method analogous thereto.

While a salt of compound (I) to be used in the present invention is not particularly limited as long as it is a pharmacologically acceptable salt, for example, inorganic acid salts such as sulfate, nitrate, hydrochloride, phosphate, hydrobromide and the like, organic acid salts such as acetate, sulfonates (e.g., p-toluenesulfonate, methanesulfonate, ethanesulfonate and the like), oxalate, maleate, fumarate, malate, tartrate, citrate, succinate, benzoate and the like can be mentioned.

As a salt of aripiprazole to be used in the present invention, those similar to the above-mentioned salts of compound (I) can be mentioned.

In the present specification, moreover, “compound (I) or a salt thereof” includes various crystal forms such as anhydride, solvate (e.g., hydrate), anhydride and solvate of compound (I) or a salt thereof, and a mixture thereof. In the present specification, moreover, “aripiprazole or a salt thereof” includes various crystal forms such as anhydride, solvate (e.g., hydrate), anhydride and solvate of aripiprazole or a salt thereof, and a mixture thereof.

The content of the medicament in the oral formulation of the present invention varies depending on the kind of the medicament, and an appropriate amount can be selected. It is generally not more than 50 wt %, preferably 0.01-50 wt %. When compound (I) or a salt thereof is used as the medicament in the present invention, the content of compound (I) or a salt thereof is preferably 0.01-20 wt %, more preferably 0.01-10 wt %, still more preferably 0.01-5 wt %. When aripiprazole or a salt thereof is used as the medicament in the present invention, the content of aripiprazole or a salt thereof is preferably 0.01-20 wt %, more preferably 0.01-10 wt %, still more preferably 0.01-5 wt %.

When a mold made of plastic, aluminum and the like is used in the production step of the oral formulation of the present invention, the quantitative ratio of respective components at the time point of filling a mixture before solidification, which is obtained by mixing and heating the respective components, into the mold (for example, when PTP (press through pack) container is used as a mold, at the time point of filling the mixture before solidification into a PTP container) does not substantially change from the quantitative ratio of the respective components in the formulation obtained by solidification, since water generally does not substantially decrease in the step of solidifying the mixture by cooling to about room temperature. In addition, since the oral formulation of the present invention is maintained in an air-tight state generally achieved by PTP packaging and the like during preservation and distribution process, the quantitative ratio of the respective components does not substantially change during such period.

The oral formulation of the present invention contains a gelling agent.

Examples of the gelling agent include gelatin, starch, pectin, carageenan, agar and the like. One or more kinds of the gelling agents can be used in combination.

From the aspects of comfortable use of the oral formulation, the gelling agent preferably contains at least gelatin (e.g., not less than 1 wt % of gelatin in gelling agent). A gelling agent containing gelatin as a main component (e.g., not less than 50 wt % of gelatin in gelling agent) is more preferable, and a gelling agent consisting solely of gelatin is further preferable.

In the present specification, “containing at least gelatin”, “gelatin as a main component” means that gelatin and other gelling agents (e.g., starch, pectin, carageenan, agar etc.) are contained as a gelling agent.

The content of the gelling agent in the oral formulation of the present invention is preferably 1-20 wt %, more preferably 1-15 wt %, still more preferably 1-12 wt %.

When the gelling agent is less than 1 wt %, the property of the formulation tends to be difficult to maintain, and when it exceeds 20 wt %, comfortableness during use tends to decrease.

The oral formulation of the present invention contains sugar alcohol.

Examples of the sugar alcohol include sorbitol, maltitol, lactitol, xylitol, erythritol, reducing paratinose, reducing starch sugar and the like. One or more kinds of sugar alcohol can be used in combination.

Sugar alcohol is a non-fermentable or decay resistant carbohydrate which can advantageously produce an oral formulation that prevents decayed teeth.

The content of sugar alcohol in the oral formulation of the present invention is preferably 50-95 wt %, more preferably 50-90 wt %, still more preferably 50-85 wt %.

When the sugar alcohol is less than 50 wt %, the comfortableness during use tends to decrease, and when it exceeds 95 wt %, the property of the formulation tends to be difficult to maintain.

As sugar alcohol in the present invention, two or more kinds selected from maltitol, sorbitol, xylitol are preferably used in combination. From the aspects of comfortableness during use, it is preferable to contain at least maltitol. Moreover, to improve comfortableness of the oral formulation during use and preservation stability, maltitol, sorbitol and xylitol are more preferably used in combination.

When maltitol, sorbitol and xylitol are used in combination, the content is, for example, maltitol 10-50 wt % (more preferably 10-40 wt %, more preferably 10-35 wt %), sorbitol 10-50 w:% (more preferably 10-40 wt %, more preferably 10-35 wt %), xylitol 10-50 wt % (more preferably 10-45 wt %, more preferably 10-40 wt %).

The mixing ratio (weight ratio) of maltitol, sorbitol and xylitol (maltitol:sorbitol:xylitol) is preferably 1:0.2-5.0:0.2-5.0, more preferably 1:0.2-3:0.2-3, still more preferably 1:0.2-2:0.2-2.

When the oral formulation of the present invention contains maltitol, sorbitol and xylitol and satisfies the above-mentioned mixing ratio, an oral formulation which shows good comfortableness during use, suppresses time-course changes in the property (hardness etc.), and has high stability during long-term preservation can be afforded.

The oral formulation of the present invention contains one or more kinds of hydrophilic polysaccharides selected from the group consisting of acacia, pullulan and maltodextrin.

These hydrophilic polysaccharides function as an agent to prevent precipitation of sugar alcohol in the present invention.

As hydrophilic polysaccharides to be used in the present invention, maltodextrin is preferable.

As maltodextrin to be used in the present invention, maltodextrin having a DE (Dextrose Equivalent) value of 5-20 is preferable, maltodextrin having a DE value of 10-20 is more preferable, and maltodextrin having a DE value of 13-20 is still more preferable.

Maltodextrin is defined as, for example, “a product in the intermediate stage, which results from hydrolysis or gelatinization of starch, and hydrolysis with acid or enzyme to give low-molecule maltose”.

As maltodextrin, a commercially available product can also be used and, for example, Pinedex #1 (DE value: 8), Pinedex #2 (DE value: 11), TK-16 (DE value: 18), Pinedex #4 (DE value: 19) (all Matsutani Chemical Industry Co., Ltd.); Amycol No. 10 (DE value: 15-16, NIPPON STARCH CHEMICAL CO., LTD.) can be mentioned.

In the oral formulation of the present invention, the content of one or more kinds of hydrophilic polysaccharides selected from the group consisting of acacia, pullulan and maltodextrin is preferably 0.1-10 wt %, more preferably 0.5-10 wt %, still more preferably 1-10 wt %.

When one or more kinds of hydrophilic polysaccharides selected from the group consisting of acacia, pullulan and maltodextrin is less than 0.1 wt %, sugar alcohol tends to precipitate, and when it exceeds 10 wt %, the property of the formulation tends to be not maintained.

The oral formulation of the present invention contains water.

The content of water in the oral formulation of the present invention is preferably 2-30 wt %, more preferably 2-25 wt %, still more preferably 5-25 wt %.

When water is less than 2 wt %, the property of the formulation tends to be difficult to maintain, and when it exceeds 30 wt %, the property of the formulation tends to be difficult to maintain or comfortableness during use tends to decrease.

A preferable embodiment of the oral formulation of the present invention is a formulation containing a medicament in an appropriate amount, 50-95 wt % of sugar alcohol, 0.1-10 wt % of one or more kinds of hydrophilic polysaccharides selected from the group consisting of acacia, pullulan and maltodextrin, 1-20 wt % of a gelling agent, 2-30 wt % of water and the below-mentioned optionally added additive (total amount being 100 wt %).

Moreover, a preferable embodiment of the oral formulation of the present invention is a formulation containing 0.01-20 wt % of compound (I) or a salt thereof (or aripiprazole or a salt thereof), 50-95 wt % of sugar alcohol, 0.1-10 wt % of one or more kinds of hydrophilic polysaccharides selected from the group consisting of acacia, pullulan and maltodextrin, 1-20 wt % of a gelling agent, 2-30 wt % of water and the below-mentioned optionally added additive (total amount being 100 wt %).

In the oral formulation of the present invention, when a basic medicament (e.g., compound (I) or a salt thereof, aripiprazole or a salt thereof) is used as the medicament, the oral formulation of the present invention preferably has pH 5-8.

In general, basic medicaments may develop a bitter taste upon dissolution. The present inventors have found that a bitter taste of the oral formulation of the present invention can be improved by setting the pH to fall within the above-mentioned range, which suppresses dissolution of the basic medicament (e.g., compound (I) or a salt thereof, aripiprazole or a salt thereof).

According to the present invention, even when a basic medicament is contained, a formulation easy to take, which can improve medication adherence and has an improved bitter taste, can be provided by adjusting the pH to the above-mentioned range.

The pH can be adjusted by a method known in the field of pharmaceutical formulation and, for example, a method using a pH adjuster can be mentioned. Examples of the pH adjuster include hydrochloric acid, phosphoric acid, carbonic acid, sulfuric acid, nitric acid, citric acid, tartaric acid, malic acid, lactic acid, acetic acid, succinic acid, maleic acid, fumaric acid, ascorbic acid, sodium citrate (e.g., monosodium citrate, disodium citrate, trisodium citrate, trisodium citrate dihydrate), calcium carbonate, sodium dihydrogen citrate, glycine, sodium tartarate, sodium hydroxide, magnesium hydroxide, sodium hydrogen carbonate, sodium carbonate, calcium lactate, sodium lactate, sodium hydrogen phosphate, sodium phosphate, calcium phosphate, meglumine and the like.

As pH adjuster to be used in the present invention, sodium citrate (e.g., monosodium citrate, disodium citrate, trisodium citrate, trisodium citrate dihydrate), calcium carbonate, sodium dihydrogen citrate, disodium citrate, glycine, sodium tartrate, sodium hydroxide, magnesium hydroxide, sodium hydrogen carbonate, sodium carbonate, calcium lactate, sodium lactate, sodium hydrogen phosphate, sodium phosphate, calcium phosphate or meglumine is preferable, trisodium citrate dihydrate is more preferable.

In the oral formulation of the present invention, an appropriate content of the pH adjuster is an amount capable of adjusting the pH to the above-mentioned range, which is generally about 0.1-5.0 wt %.

The oral formulation of the present invention may contain a pharmaceutically acceptable additive as necessary such as colorant, flavor, preservative and the like.

Examples of the colorant include red cabbage (red), safflower yellow (yellow), gardenia blue (blue), iron oxide (e.g., red ferric oxide, yellow ferric oxide), aluminum lake, caramel, β-carotene, various food colors (Food Color yellow No. 1, Food Color Red No. 2 etc.) and the like.

Examples of the preservative include benzoic acid, sodium benzoate, sodium sorbate, methyl p-hydroxybenzoate, propyl p-hydroxybenzoate and the like.

Examples of the flavor include orange flavor, passion fruit flavor, strawberry flavor, cherry flavor, apple flavor, lemon flavor, grape flavor, coffee flavor, black tea flavor, herb mint flavor, chocolate flavor and the like.

The oral formulation of the present invention has a gummy-like comfortableness during use.

In the present specification, “gummy” generally refers to a gel composition wherein a composition mainly composed of carbohydrates and water is gelled by a gelling agent, and is a concept encompassing confectionery widely known as gummy, gummy candy and the like.

The oral formulation of the present invention can be produced, for example, by the following method.

Sugar alcohol (for example, maltitol, sorbitol, xylitol etc.) and purified water are mixed and dissolved by heating. Thereto is added a medicament (for example, compound (I) or a salt thereof, aripiprazole or a salt thereof etc.) and the mixture is mixed by stirring with heating to uniformity. The sugar alcohol solution, a gelling agent (for example, gelatin etc.) swollen in advance with purified water, a pH adjuster (for example, sodium citrate etc.) and hydrophilic polysaccharides are added, and the mixture is mixed by stirring with heating. To the mixture is added an optionally added additive (for example, flavor etc.) and the mixture is further mixed by stirring with heating to give a medicament-containing mixture (mixture before solidification). The medicament-containing mixture is solidified by cooling to give an oral formulation.

In the above-mentioned method, the step of solidifying the medicament-containing mixture by cooling is performed, for example, as shown below.

The medicament-containing mixture is filled in a container obtained by forming a concave in a plastic sheet of vinyl chloride and the like or an aluminum sheet, the medicament-containing mixture is left standing to allow solidification, whereby an oral formulation can be obtained. Where necessary, a mold lubricant such as medium-chain triglyceride and the like can also be applied to the inside of the container. The mold lubricant may contain a glidant such as light anhydrous silicic acid, talc, magnesium stearate and the like as necessary. This method is advantageous in that the plastic or aluminum container can be directly handled as a PTP package.

The oral formulation of the present invention can be orally administered to human safely. Preferably, it is administered without water by being licked or crunched in the mouth.

The oral formulation of the present invention containing compound (I) or a salt thereof (or aripiprazole or a salt thereof) can be used for treating CNS (Central Nervous System)-related disorders such as schizophrenia, depression, bipolar disorder, dementia and the like in human patients.

The dose of the oral formulation of the present invention varies depending on the kind of the medicament, kind and severity of the disease and the like. When compound (I) or a salt thereof (or aripiprazole or a salt thereof) is used as a medicament, the dose is generally 0.05-50 mg as compound (I) or a salt thereof (or aripiprazole or a salt thereof) per day.

The size and shape of the oral formulation of the present invention is not particularly limited. For example, an oral formulation generally having a weight of about 300-10000 mg, particularly about 500-6000 mg, per formulation can be mentioned.

As a package form of the oral formulation of the present invention, packaging in an airtight container is preferable and, for example, PTP package (e.g., aluminum PTP package) can be mentioned.

In addition, the present invention also relates to a substrate for oral formulation, which contains sugar alcohol; one or more kinds of hydrophilic polysaccharides selected from the group consisting of acacia, pullulan and maltodextrin; a gelling agent; and water.

Examples and content of each component (sugar alcohol; one or more kinds of hydrophilic polysaccharides selected from the group consisting of acacia, pullulan and maltodextrin; a gelling agent; and water) are those similar to the examples and content explained for the above-mentioned oral formulation of the present invention.

The substrate for the oral formulation of the present invention can be produced by the above-mentioned production method of the oral formulation of the present invention except that the medicament is absent, or a method analogous thereto.

### EXAMPLES

The present invention is explained in more detail in the following by referring to Examples and Experimental Examples, which are not to be construed as limitative.

### Examples 1-8

According to the compounding ratios shown in Table 1, sugar alcohol (maltitol, sorbitol, xylitol) and purified water were mixed, and the mixture was dissolved by heating at about 140° C. Compound (I) was added and the mixture was mixed by stirring to uniformity. Gelatin was swollen with purified water containing trisodium citrate dihydrate, maltodextrin, methyl p-hydroxybenzoate and propyl p-hydroxybenzoate, dissolved by heating at about 70° C. and added to the medicament-containing sugar alcohol mixture, and the mixture was mixed by stirring. To the mixture were added red ferric oxide, yellow ferric oxide and flavor, and the mixture was further mixed by stirring to give a medicament-containing mixture having a mixing ratio shown in Table 1. The obtained medicament-containing mixture was filled in an aluminum PTP container at 750 mg per container and solidified by cooling at room temperature for 24 hr or longer to give the oral formulations of Examples 1-6.

In the same manner as in Examples 1-6 except that aripiprazole was used as a medicament according to the compounding ratios shown in Table 1, the oral formulation of Example 7 was obtained.

In the same manner as in Examples 1-6 except that the medicament was not used according to the compounding ratios shown in Table 1, the substrate for oral formulation of Example 8 was obtained.

The oral formulations of Examples 1-7 and the substrate for oral formulation of Example 8 were confirmed to have pH 5-8 by pH test paper.

TABLE 1

| mg/unit | Example 1 | Example 2 | Example 3 | Example 4 | Example 5 | Example 6 | Example 7 | Example 8 |
|---|---|---|---|---|---|---|---|---|
| compound (I) | 4 | 4 | 4 | 4 | 4 | 4 | 0 | 0 |
| aripiprazole | 0 | 0 | 0 | 0 | 0 | 0 | 3 | 0 |
| gelatin | 90 | 90 | 60 | 60 | 75 | 75 | 60 | 60 |
| maltitol | 120 | 138.75 | 135 | 150 | 105 | 120 | 150 | 150 |
| sorbitol | 120 | 138.75 | 210 | 210 | 165 | 195 | 210 | 210 |
| xylitol | 195 | 195 | 225 | 225 | 225 | 210 | 225 | 225 |
| maltodextrin | 75 | 37.5 | 30 | 15 | 60 | 30 | 15 | 15 |
| trisodium citrate dihydrate | 4 | 4 | 4 | 4 | 4 | 4 | 4 | 4 |
| methyl p-hydroxybenzoate | 0.150 | 0.150 | 0.090 | 0.090 | 0.120 | 0.120 | 0.180 | 0.180 |
| propyl p-hydroxybenzoate | 0.0225 | 0.0225 | 0.0135 | 0.0135 | 0.0180 | 0.0180 | 0.0300 | 0.0300 |
| red ferric oxide | 0.0225 | 0.0225 | 0.0225 | 0.0225 | 0.0225 | 0.0225 | 0.0225 | 0.0225 |
| yellow ferric oxide | 0.1275 | 0.1275 | 0.1275 | 0.1275 | 0.1275 | 0.1275 | 0.1275 | 0.1275 |
| flavor | 0.75 | 0.75 | 0.75 | 0.75 | 0.75 | 0.75 | 0.60 | 0.60 |
| water | 140.93 | 140.93 | 81.00 | 81.00 | 110.96 | 110.96 | 82.04 | 85.04 |
| total | 750 | 750 | 750 | 750 | 750 | 750 | 750 | 750 |

### Experimental Example 1

The substrate for oral formulation of Example 8 was evaluated for easy administration by the following 3 criteria by chewing in the mouth by reference to the hardness of commercially available gummy as the standard. As a result, the administrability was good.

good: good hardness
rather bad: somewhat insufficient hardness
bad: soft

### Experimental Example 2

According to the compounding ratios shown in Table 2, sugar alcohol (maltitol, sorbitol) and purified water were mixed, and the mixture was dissolved by heating at about 140° C. and boiled down to an optional water content. Gelatin was swollen with purified water containing trisodium citrate dihydrate and maltodextrin, dissolved by heating at about 70° C. and added to the sugar alcohol solution, and the mixture was mixed by stirring to give a mixed solution having a mixing ratio shown in Table 2. The obtained mixed solution was filled in an aluminum PTP container at 750 mg per container and solidified by cooling at room temperature for 24 hr or longer to give the substrates for oral formulations of Examples 9 and 10.

In the same manner as in Examples 9 and 10 except that maltodextrin was not added according to the compounding ratios shown in Table 2, the substrate of Comparative Example 1 was obtained.

The substrates for oral formulations of Examples 9 and 10 and Comparative Example 1 were confirmed to have pH 5-8 by pH test paper.

Each of the obtained substrates was preserved at 40° C. for 3 weeks. As a result, the substrate of Comparative Example 1 without containing maltodextrin showed precipitation of sugar alcohol but precipitation was not seen in the substrates of Examples 9 and 10 containing maltodextrin.

TABLE 2

| | | | Comparative |
|---|---|---|---|
| formulation % | Example 9 | Example 10 | Example 1 |
| gelatin | 6 | 6 | 6 |
| maltitol | 38 | 38 | 40 |
| sorbitol | 38 | 38 | 40 |
| maltodextrin (*1) | 4 | 0 | 0 |
| maltodextrin (*2) | 0 | 4 | 0 |
| trisodium citrate | 0.5 | 0.5 | 0.5 |
| dihydrate | | | |
| water | 13.5 | 13.5 | 13.5 |
| total | 100 | 100 | 100 |

(*1): TK-16 (trade name, Matsutani Chemical Industry Co., Ltd.)
(*2): Amycol No. 10 (trade name, NIPPON STARCH CHEMICAL CO., LTD.)

### INDUSTRIAL APPLICABILITY

According to the present invention, an oral formulation that can be taken easily even without water and can improve medication adherence, and a substrate for oral formulation can be provided.

This application is based on U.S. provisional patent application Nos. 61/640,474 and 61/783,163, the contents of which are incorporated in full herein.

1. An oral formulation comprising a medicament; sugar alcohol; one or more hydrophilic polysaccharides selected from the group consisting of acacia, pullulan and maltodextrin; a gelling agent; and water.

2. The oral formulation according to claim 1, wherein the sugar alcohol comprises one or more of maltitol, sorbitol, or xylitol.

3. The oral formulation according to claim 2, wherein the sugar alcohol comprises maltitol, sorbitol, and xylitol.

4. The oral formulation according to claim 1, wherein the one or more hydrophilic polysaccharides comprises at least maltodextrin.

5. The oral formulation according to claim 1, wherein the content of one or more hydrophilic polysaccharides is 0.1-10 wt %.

6. The oral formulation according to claim 1, wherein the gelling agent comprises at least gelatin.

7. The oral formulation according to claim 1, wherein the content of water is 2-30 wt %.

8. The oral formulation according to claim 1, wherein the content of sugar alcohol is 50-95 wt %.

9. The oral formulation according to claim 1, wherein the content of the gelling agent is 1-20 wt %.

10. The oral formulation according to claim 1, wherein the gelling agent consists only of gelatin.

11. The oral formulation according to claim 1, wherein the medicament is a basic medicament.

12. The oral formulation according to claim 11, wherein the basic medicament is 7-[4-(4-benzo [b] thiophen-4-yl-piperazin-I-yl) butoxy]-IH-quinolin-2-one or a salt thereof, or aripiprazole or a salt thereof.

13. The oral formulation according to claim 1, further comprising one or more additives selected from the group consisting of a flavor, a colorant, a preservative, and a pH adjuster.

14. The oral formulation according to claim 1, wherein the pH is adjusted to 5-8.

15. A substrate for oral formulation, comprising sugar alcohol; one or more hydrophilic polysaccharides selected from the group consisting of acacia, pullulan, and maltodextrin; a gelling agent; and water.
